(19) United States
(12) Patent Application Publication
(10) Pub. No.: US 2011/0054275 A1
(43) Pub. Date: Mar. 3, 2011

(54) Mounting Unit Having a Sensor and Associated Circuitry

(75) Inventor: Gary Ashley Stafford, Hayward, CA (US)

(73) Assignee: Abbott Diabetes Care Inc., Alameda, CA (US)

(21) Appl. No.: 12/873,301

(22) Filed: Aug. 31, 2010

Related U.S. Application Data
(60) Provisional application No. 61/238,494, filed on Aug. 31, 2009.

(51) Int. Cl. A61B 5/00

(52) U.S. Cl. 600/309

(57) ABSTRACT

Disclosed herein is a mounting unit assembly that includes a mounting unit and a sensor integrally formed with the mounting unit such that the mounting unit and sensor form a single unit.

### RELATED APPLICATION

The present application claims the benefit of U.S. Provisional Patent Application No. 61/238,494, entitled “A Mounting Unit Having Sensor and Circuitry”, filed on Aug. 31, 2009, the disclosure of which is incorporated herein by reference in its entirety for all purposes.

### BACKGROUND

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type I Diabetes) or does not properly utilize insulin (a condition known as Type II Diabetes), the blood glucose remains in the blood resulting in hyperglycemia or abnormally high blood sugar levels.

The vast and uncontrolled fluctuations in blood glucose levels in people suffering from diabetes cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

The first step in managing blood glucose levels is testing and monitoring blood glucose levels by using conventional techniques, such as drawing blood samples, applying the blood to a test strip, and determining the blood glucose level using colorimetric, electrochemical, or photometric test meters. Another more recent technique for monitoring blood glucose levels is by using a continuous or automatic glucose monitoring system.

Unlike conventional blood glucose meters, continuous analyte monitoring systems employ an insertable or implantable sensor, which have sensor electrochemistry configured to continuously detect and monitor blood glucose levels. The glucose signals are then transmitted by a transmitter to a receiver unit. In most cases, the transmitter is a separate unit having its own electronic circuitry to process the data from the sensor and to transmit the processed data to a receiver. The transmitter is usually powered up by a power source contained within the transmitter body, thus, the transmitter body must be sufficiently sized to include both the electronic circuitry and power source.

The implantable nature of the sensor and the continuous monitoring necessitate that the sensor and the transmitter be components of an on-body device. Thus, the size of both the transmitter and sensor can create comfort issues for the user who wears these devices. Accordingly, there is a need for mounting assemblies to have sensors and transmitters that are compact and have reduced profiles to improve comfort for the user.

### INCORPORATED BY REFERENCE

The following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,041,468; 7,167,818; and 7,299,082; U.S. Published Application Nos. 2004/0186365; 2005/0182306; 2006/0025662; 2006/0091006; 2007/0056858; 2007/0068807; 2007/0078322; 2007/0095661; 2007/0108048; 2007/0199818; 2007/0227911; 2007/0233013; 2008/0066305; 2008/0081977; 2008/0102441; 2008/0148873; 2008/0161666; 2008/0267823; and 2009/0054748; U.S. patent application Ser. Nos. 11/461,725; 12/131,012; 12/393,921, 12/242,823; 12/363,712; 12/495,709; 12/698,124; 12/698,129; 12/714,439; 12/794,721; and 12/842,013, and U.S. Provisional Application Nos. 61/317,243, 61/345,562, and 61/361,374.

### SUMMARY

The present disclosure is generally directed to a mounting unit assembly. The mounting unit assembly generally includes a body having one or more contacts and a sensor disposed on or formed into one of the surfaces of the body such that the sensor and mounting unit form a single unitary structure. The body also includes one or more conductive traces disposed along a length thereof. In certain embodiments, the one or more conductive traces extend from the sensor to the contacts disposed on the mounting unit body. Various methods can be used to associate the one or more contacts and the conductive traces with the mounting unit body. For example, the contacts and/or conductive traces can be embedded, molded, or etched into or onto the mounting unit body. Further, the sensor can be embedded, molded or etched into or onto the mounting unit body. In still yet another embodiment, the conductive traces and contacts may be formed from a flexible ribbon disposed on mounting unit body. In another embodiment, the mounting unit can be configured to include a sensor integral with the mounting unit body. For example, a portion of the mounting unit body can be configured with sensor electrochemistry to detect and monitor an analyte of interest.

In certain embodiments, the mounting unit assembly further includes a transmitter having one or more transmitter contacts disposed on a surface thereof. The one or more transmitter contacts are configured to interface with the one or more contacts disposed on the mounting unit body so as to establish electrical conductance.

The contacts and one or more conductive traces can be formed from conductive material, such as, but not limited to, a conductive polymer, conductive metal, or carbon. For example, the conductive metal can be gold. In some embodiments, gold contacts and gold conductive traces are formed by laser ablation techniques.

In certain embodiments, the sensor body includes a substrate, a sensing layer, and a barrier layer. The sensor body can have a proximal end and a distal end having different widths. In such embodiments, the distal end defines a sensor tail configured for implantation in the body of a user. In some instances, the sensor tail can be configured to project downwardly from a surface of the mounting unit.

In still yet other embodiments, the mounting unit includes a power source, such as, for example, a battery. In this regard, the transmitter can be powered by the battery disposed in the mounting unit body when the transmitter is operatively coupled to the mounting unit body.

In another aspect, a sensor insertion mechanism is provided. The sensor insertion mechanism includes a housing, an introducer sharp movably coupled within the housing, and an actuator for urging the introducer sharp in an insertion direction. The introducer sharp can be configured to releasably receive at least a portion of a sensor. For example, the portion of the sensor can be slideably received within the introducer sharp.

In certain embodiments, the mounting unit includes a sensor associated with the mounting unit body. As such, the sensor can be secured to the mounting unit and engage the introducer sharp. For example, the sensor can have a thermoformed body configured to fit within a channel disposed in the introducer sharp. In yet another embodiment, the sensor can have a body configured to slideably engage with the introducer sharp.

In certain embodiments, the actuator of the sensor insertion mechanism is a button, such as, for example, a dome or membrane. Additionally, the actuator can include a spring to urge the introducer sharp back to an insertion position once manual force has been applied to the button.

In one aspect, the insertion mechanism is removable from the mounting unit body. For example, after insertion of the sensor, the introducer sharp is configured to retract back into the housing of the insertion mechanism, for example, by way of a retraction spring. After insertion of the sensor and removal of the insertion mechanism from the mounting unit, a transmitter can be engaged to the mounting unit. According to embodiments, the sensor maintains a substantially flat configuration when the transmitter is engaged with the mounting unit.

These and other objects, features and advantages of the present disclosure will become more fully apparent from the following detailed description of the embodiments, the appended claims and the accompanying drawings.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 illustrates a block diagram of a data monitoring and management system according to embodiments of the present disclosure;

FIG. 2 is a schematic view of mounting unit assembly according to embodiments of the present disclosure;

FIGS. 3A to 3C are schematic views of a sensor insertion mechanism according to embodiments of the present disclosure; and

FIG. 4 is a schematic view of a mounting unit assembly according to embodiments of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Various exemplary embodiments of the analyte monitoring system and methods of the disclosure are described in further detail below. Although the disclosure is described primarily with respect to a glucose monitoring system, each aspect of the disclosure is not intended to be limited to the particular embodiment so described. Accordingly, it is to be understood that such description should not be construed to limit the scope of the disclosure, and it is to be understood that the analyte monitoring system can be configured to monitor a variety of analytes, as described below.

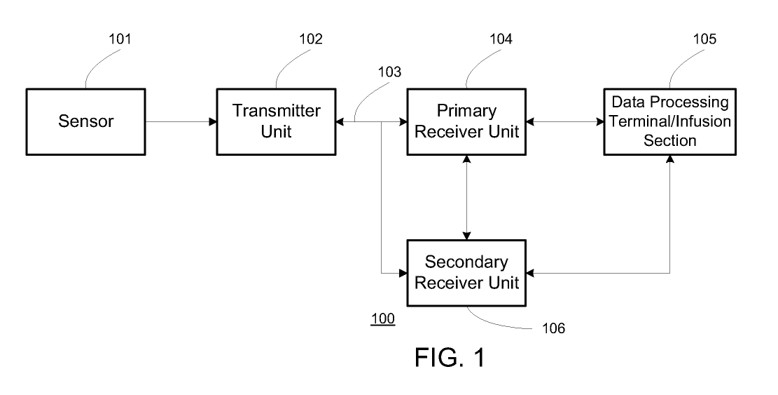

Embodiments of the present disclosure are directed to a mounting unit assembly for use with an analyte monitoring system such as, for example, a continuous, semi-continuous, or a discrete glucose monitoring system where continuous or semi-continuous monitoring systems include sampling or detection of a glucose level according to a programmed or programmable schedule or time intervals, while discrete glucose monitoring systems determine a glucose level at discrete times, for example, when a user wishes to know that information. An exemplary analyte monitoring system that can be used with the mounting unit assembly is represented in FIG. 1.

FIG. 1 illustrates a data monitoring and management system such as, for example, analyte (e.g., glucose) monitoring system 100 in accordance with embodiments of the present disclosure. The analyte monitoring system 100 includes a sensor 101, a transmitter unit 102 coupleable to the sensor 101, and a primary receiver unit 104 which is configured to communicate with the transmitter unit 102 via a bi-directional communication link 103. The primary receiver unit 104 may be further configured to transmit data to a data processing terminal 105 for evaluating the data received by the primary receiver unit 104. Moreover, the data processing terminal 105 in one embodiment may be configured to receive data directly from the transmitter unit 102 via a communication link which may optionally be configured for bi-directional communication. Accordingly, transmitter unit 102 and/or receiver unit 104 may include a transceiver.

Also shown in FIG. 1 is an optional secondary receiver unit 106 which is operatively coupled to the communication link and configured to receive data transmitted from the transmitter unit 102. Moreover, as shown in the Figure, the secondary receiver unit 106 is configured to communicate with the primary receiver unit 104 as well as the data processing terminal 105. Indeed, the secondary receiver unit 106 may be configured for bidirectional wireless communication with each or one of the primary receiver unit 104 and the data processing terminal 105. In one embodiment of the present disclosure, the secondary receiver unit 106 may be configured to include a limited number of functions and features as compared with the primary receiver unit 104. As such, the secondary receiver unit 106 may be configured substantially in a smaller compact housing or embodied in a device such as a wrist watch, pager, mobile phone, Personal Digital Assistant (PDA), for example. Alternatively, the secondary receiver unit 106 may be configured with the same or substantially similar functionality as the primary receiver unit 104. The receiver unit may be configured to be used in conjunction with a docking cradle unit, for example for one or more of the following or other functions: placement by bedside, for re-charging, for data management, for night time monitoring, and/or bidirectional communication device.

In one aspect analyte monitoring system 100 may include two or more sensors, each configured to communicate with transmitter unit 102. Furthermore, while only one, transmitter unit 102, communication link 103, and data processing terminal 105 are shown in the embodiment of the analyte monitoring system 100 illustrated in FIG. 1, in certain embodiments, the analyte monitoring system 100 may include one or more sensors, multiple transmitter units 102, communication links 103, and data processing terminals 105. Moreover, within the scope of the present disclosure, the analyte monitoring system 100 may be a continuous monitoring system, or semi-continuous, or a discrete monitoring system. In a multi-component environment, each device is configured to be uniquely identified by each of the other devices in the system so that communication conflict is readily resolved between the various components within the analyte monitoring system 100.

In one embodiment of the present disclosure, sensor 101 is physically positioned in or on the body of a user whose analyte level is being monitored. The sensor 101 may be configured to continuously sample the analyte level of the user and convert the sampled analyte level into a corresponding data signal for transmission by the transmitter unit 102. In certain embodiments, the transmitter unit 102 may be physically coupled to the sensor 101 so that both devices are integrated in a single housing and positioned on the user's body. The transmitter unit 102 may perform data processing such as filtering and encoding on data signals and/or other functions, each of which corresponds to a sampled analyte level of the user, and in any event transmitter unit 102 transmits analyte information to the primary receiver unit 104 via the communication link 103. Additional detailed description of the continuous analyte monitoring system, its various components including the functional descriptions of the transmitter are provided in but not limited to U.S. Pat. Nos. 6,134,461, 6,175,752, 6,121,611, 6,560,471, and 6,746,582, and U.S. Patent Publication No. 2008/0278332 and elsewhere, the disclosures of each of which are incorporated by reference for all purposes.

As described above, the sensor 101 is configured to be physically positioned in or on the body of the user. Additionally, in certain embodiments, the transmitter unit 102 is coupled to the sensor 101. In certain embodiments, a connection between contact portions of a transmitter unit 102 and contact portions the sensor 101 is dependent on the placement or alignment of the sensor 101. For example, a sensor 101 may be inserted into the body of a user and become skewed or tilted prior to being coupled to the transmitter unit 102. As result of the sensor being tilted or skewed, the sensor 101 may not properly align with the transmitter unit 102 which may result in sensor data not being transmitted from the transmitter unit 102 to the primary receiver unit 104. Accordingly, in certain embodiments, a mounting unit is provided in which the sensor 101 is physically located on the mounting unit such that when the transmitter unit 102 is coupled to the mounting unit, the connection between the transmitter unit 102 and the sensor 101 is not dependent sensor placement or alignment.

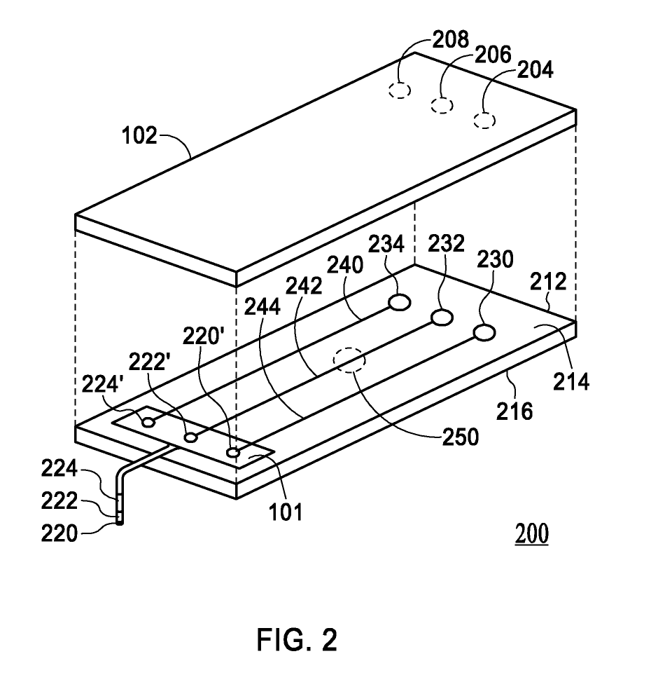

FIG. 2 is a schematic view of a mounting unit 200 according to embodiments of the present disclosure. Referring to FIG. 2, in certain embodiments, mounting unit 200 includes a mounting unit body 212 having first and second opposing surfaces (upper surface 214 and lower surface 216). In certain embodiments, and as will be described in greater detail below, a sensor, such as for example sensor 101 (FIG. 1) and electronic circuitry are designed so as to be directly on one or more surfaces of the mounting unit body 212. In certain embodiments, the mounting unit body 212 has a generally planar configuration including a rectangular configuration having first and second ends. Although a rectangular configuration has been specifically mentioned, it is contemplated that the mounting unit body 212 can have other configurations, such as, for example, a circular configuration or a polygonal configuration.

Various materials can be employed to form the mounting unit body 212. For example, in certain embodiments, the mounting unit body 212 can be formed from flexible or rigid materials. Suitable materials for a flexible body may include but are not limited to non-conducting plastic or polymeric materials and other non-conducting, flexible, deformable materials. Suitable plastic or polymeric materials include thermoplastics such as polycarbonates, polyesters (e.g., Mylar® and polyethylene terephthalate (PET)), polyvinyl chloride (PVC), acrylonitrile butadiene styrene (ABS), polyurethanes, polyethers, polyamides (e.g., nylons), polyimides, styrene, delrin, carbon fiber, or copolymers of these thermoplastics, such as PETG (glycol-modified polyethylene terephthalate). Suitable materials for a rigid body include but are not limited to poorly conducting ceramics, such as aluminum oxide and silicon dioxide. Further, mounting unit body 212 can be formed from other materials, such as insulating materials. Suitable insulating materials include, but are not limited to, polyurethane, teflon (fluorinated polymers), polyethyleneterephthalate (PET, Dacron) or polyimide.

Referring back to FIG. 2, in certain embodiments, sensor 101 is formed in or on the first surface 214 of the mounting unit body 212. In this regard, a body of the sensor 101 is affixed or otherwise permanently secured on or into the mounting unit body 212 by various techniques. For example, the sensor 101 can be embedded, adhered, etched or molded on or into the mounting unit body 212 with at least a portion of the sensor 101 extending from the mounting unit body 212. In certain embodiments, the portion of the sensor 101 extending from the mounting unit body 212 is an insertion portion of the sensor 101. Because the sensor 101 is integrally formed within the mounting unit body 212, the sensor 101 will not become skewed or misaligned when the insertion portion of the sensor 101 is inserted into the skin of a user. As a result, when a transmitter is coupled to the mounting unit body 212, the contact points on the transmitter unit 102 may be more easily aligned with the contact points on the sensor 101 and/or mounting unit body 212 thus establishing a more reliable connection between the transmitter unit 102 and sensor 101.

As described above, sensor 101, and electronic components associated with the sensor 101 (e.g. traces, capacitors, etc.), may be embedded directly into the mounting unit body 212. For example, portions of the sensor 101 including sensor electronics and/or chemistry associated with the sensor 101 are disposed on or otherwise directly connected to the mounting unit body 212. Thus, in certain embodiments, the mounting unit body 212 and the sensor 101 form a single unitary structure, such that mounting unit body 212 is disposed along with sensor 101 after the useful sensor life of sensor 101.

In certain embodiments, the sensor 101 comprises a substrate and one or more electrodes disposed thereon. For example, in certain embodiments, sensor 101 includes three electrodes, such as a working electrode 120, counter electrode 122, and a reference electrode 124 that are disposed near the distal end of the sensor 101. The sensor 101 can further include conductive traces (not shown) extending from the electrodes 220, 222, 224 to corresponding, respective contacts 220′, 222′, 224′ to define the sensor 101 electronic circuitry. As described above, at least a portion of the conductive traces and the contacts 220′, 222′, 224′ are embedded, adhered, etched or molded directly into or on the mounting unit body 212. Although specific examples are given, it is contemplated that fewer or more electrodes may be used with the sensor 101. Additional detailed description of sensors and various components of sensors including the functional descriptions of each of the components are provided in but not limited to U.S. Pat. Nos. 6,103,033, 6,134,461, and 6,175,752 and U.S. patent application Ser. No. 12/393,921 and elsewhere, the disclosures of each of which are incorporated herein by reference for all purposes.

Referring back to FIG. 2, in certain embodiments, the mounting unit body 212 further includes one or more contacts 230, 232, 234 and/or conductive traces 240, 242, 244 disposed along a length of the mounting unit body 212. As with the sensor electronics described above, the contacts 230, 232, 234 and/or the conductive traces 240, 242, 244 are molded, embedded, or etched directly onto the mounting unit body 212, such as, for example the upper surface 214 of the mounting unit body 212. Because some of the electronic components described above are formed directly into the mounting unit body 212, a transmitter such as, for example, transmitter unit 102 (FIG. 1) may require fewer electronic components to receive and transmit a signal from the sensor 101. As a result, in certain embodiments, the transmitter unit 102 may have a lower profile because it does not contain the conductive traces and other electronic components described above.

In certain embodiments, the electronic circuitry associated with the mounting unit 200 can be, for example, formed from one or more conductive materials. Likewise, the electrodes, conductive traces, and contacts disposed on the sensor 101 can be formed from one or more conductive materials. Suitable conductive materials include, but are not limited to, vitreous carbon, graphite, silver, silver-chloride, platinum, palladium, platinum-iridium, or gold. The conductive material can be applied to the mounting unit body and/or the sensor by various techniques including laser ablation, printing, etching, and photolithography.

In certain embodiments, contacts 230, 232, 234 disposed on the upper surface 214 of the mounting unit body 212 are configured to interface directly with contacts 204, 206, 208 that have been insert molded into a transmitter such as, for example transmitter unit 102 (FIG. 1). As the contacts 204, 206, 208 are insert molded into the transmitter unit 102, the contacts are leak proof, dust proof, and air tight and do not require a separate assembly step when the transmitter unit 102 is being manufactured. In one aspect, the contacts 204, 206, 208 are made of metal or a conductive polymer such that when the contacts are compressed, the contacts 204, 206, 208 expand and form a larger surface area for connection with the mounting unit contacts 230, 232, 234. In another embodiment, the transmitter contacts 204, 206, 208 may be inserted directly into the mounting unit contacts 230, 232, 234 such that the transmitter contacts fit at least partially within the mounting unit contacts 230, 232, 234 when the transmitter 102 is coupled to the mounting unit body 212.

In certain embodiments, mounting unit electronic circuitry, (e.g., the one or more contacts and one or more conductive traces disposed on the mounting unit body 212), may also include RF communication transmission capacitors for transmission of analyte data to an external receiver unit such as, for example primary receiver unit 104. Additionally, an antenna, battery, integrated circuit, and/or other passive electronics can also be associated with the mounting unit body 212.

In certain embodiments, the mounting unit body 212 also includes an application specific integrated circuit (ASIC) in electrical contact with a plurality of RF communication transmission capacitors positioned, for example, substantially around the outer periphery of the a flexible circuit board disposed on the mounting unit body 212. In such embodiments, the RF communication transmission link is configured for bi-directional or uni-directional communication, as described above in relation to sensor 101 and transmitter 102.

Referring back to FIG. 2, in certain embodiments, the mounting unit 200 can further include a temperature module 250 disposed on lower surface 216 of the mounting unit body 212. It is contemplated that the temperature module 250 may be placed in variable positions on the lower surface 216 of the mounting unit body 212 depending on a desired configuration. In certain embodiments, the temperature module 250 is configured to measure the temperature of the user's skin surrounding the sensor insertion site. As the temperature of the user's skin changes, the signal from the temperature module 250 (e.g., the amount of current flowing through the probe) may be combined with the signal obtained from one of the electrodes (e.g., the working electrode 120) by, for example, scaling the temperature probe signal and then adding or subtracting the scaled temperature module signal from the signal at the working electrode 120. In this manner, the temperature module 250 can provide a temperature adjustment for the output from the working electrode 120.

In certain embodiments, a temperature module such as described herein may be formed using two probe leads connected to each other through a temperature-dependent element formed using a material with a temperature-dependent characteristic. The two probe leads of the temperature module can be formed using a metal, an alloy, a semimetal, such as graphite, a degenerate or highly doped semiconductor, or a small-band gap semiconductor. Some non-limiting examples of suitable materials include gold, silver, ruthenium oxide, titanium nitride, titanium dioxide, indium doped tin oxide, tin doped indium oxide, or graphite.

In certain embodiments, the temperature-dependent element is typically made using a fine trace (e.g., a conductive trace that has a smaller cross-section than that of the probe leads) of the same conductive material as the probe leads, or another material such as a carbon ink, a carbon fiber, or platinum, which has a temperature-dependent characteristic, such as resistance, that provides a temperature-dependent signal when a voltage source is attached to the two probe leads of the temperature module. The temperature-dependent characteristic of the temperature-dependent element may either increase or decrease with temperature. Preferably, the temperature dependence of the characteristic of the temperature-dependent element is approximately linear with temperature over the expected range of biological temperatures (about 25 to 45° C.), although this is not required.

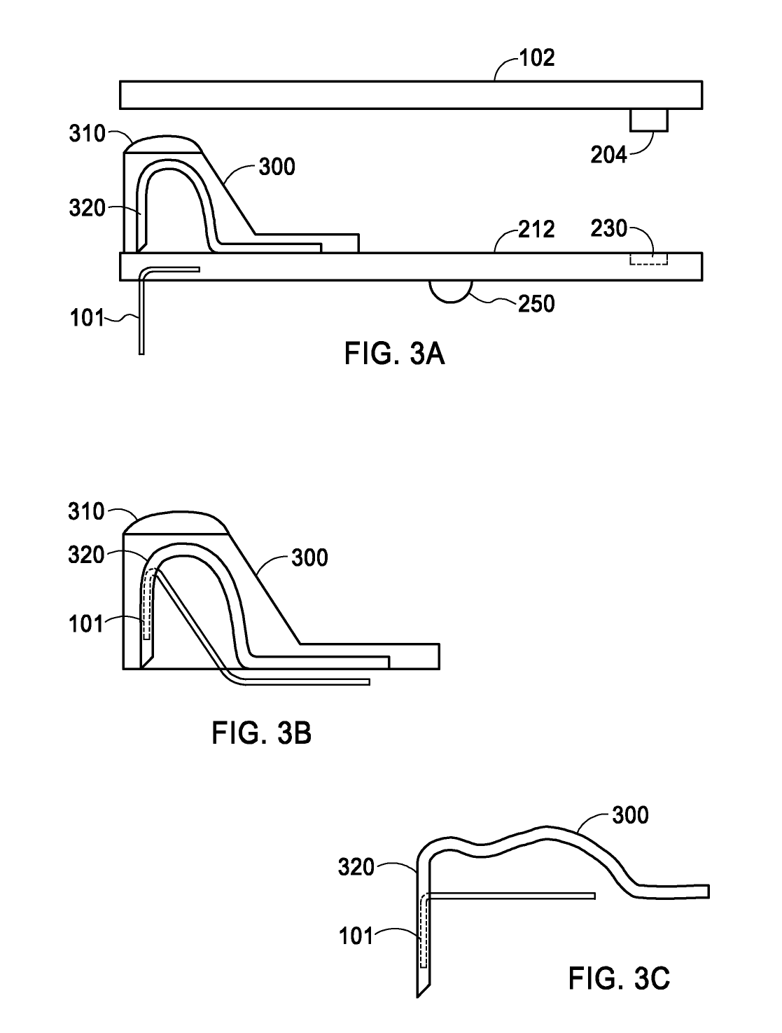

In certain embodiments, at least a portion of the sensor 101 may be configured to be inserted into a skin layer of a user. Referring to FIG. 3A, a sensor insertion mechanism 300 is provided, in certain embodiments, to assist a user with inserting the insertion portion of the sensor 101 into subcutaneous tissue of a user. In certain embodiments, the sensor insertion mechanism 300 is removeably attached to the mounting unit body 212 by, for example, a snap-on interface, sliding mechanism (e.g., tracks on a portion of the mounting unit body 212 such that the insertion mechanism may slideably move along the tracks) or other fastening member. Although a snap-on interface is specifically mentioned, it is contemplated that other temporary fastening mechanisms may be used, such as adhesives, connection pins and the like.

In certain embodiments, the sensor insertion mechanism 300 includes an introducer sharp 320 and an actuator 310 configured to actuate insertion of at least a portion of the sensor 101 into a skin layer of a user. In certain embodiments, the introducer sharp 320 is configured to receive the insertion portion of the sensor 101 as is shown in FIG. 3B prior to the insertion portion of the sensor 101 being inserted into the skin of the user. In certain embodiments, the introducer sharp 320 is a polished needle having a U-shaped configuration which enables the insertion portion of the sensor 101 to slideably engage with the introducer sharp 320. In certain embodiments, the introducer sharp 320 is a spring, such as, for example, a flexible flat spring configured to engage the insertion portion of the sensor 101. In still yet other embodiments, the insertion portion of the sensor 101 can be thermoformed to fit within a channel formed in a longitudinal axis of the introducer sharp 320.

In certain embodiments, a manual force is applied to the introducer sharp 320 via an actuator or button 310, and the introducer sharp 320 moves in a downward direction toward the user's skin. The introducer sharp 320 pierces the user's skin and the insertion portion of the sensor 101 that is disposed within the introducer sharp 320 is implanted in the skin. Upon release of the manual pressure, the introducer sharp 320 releases insertion portion of the sensor 101 and is automatically withdrawn back into the sensor insertion mechanism 300.

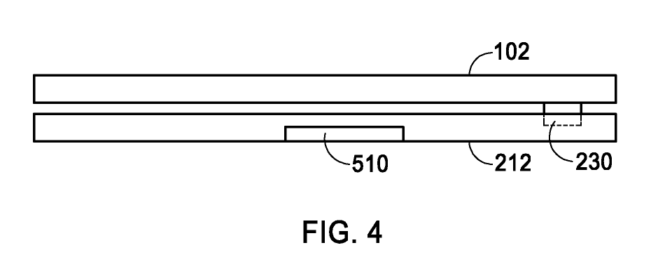

As illustrated in FIG. 4C, when manual force is applied to the actuator 310, the actuator 310 is depressed and propels the introducer sharp 320 and the insertion portion of the sensor 101 downwardly to an insertion position, (e.g., into the skin of the user). After insertion, the introducer sharp 320 automatically retracts, for example, by a return spring, leaving the insertion portion of the sensor 101 at least partially below the skin while the attached mounting unit body 212 remains on the surface of the skin. In certain embodiments, at least a portion of the introducer sharp 320 may include the retraction spring such that the introducer sharp 320 withdraws itself from the skin of the user when the manual force is removed from the actuator 310. In certain embodiments, the actuator 310 may be made of a spring like material and may be at least partially coupled to the introducer sharp 320. Thus, as the manual force is removed from the actuator 310, the actuator 310 returns to its original shape which in turn, pulls the introducer sharp 320 from the skin. In certain embodiments, the introducer sharp 320 is entirely contained within the sensor insertion mechanism 300 once it has been withdrawn from the skin of the user.

In still yet another embodiment, the insertion portion of the sensor 101 may be sufficiently rigid such that the insertion portion of the sensor 101 may be inserted through the skin layer of the user without using an introducer sharp 320 as was described above. In such cases, the actuator 310 may be operatively coupled to the insertion portion of the sensor 101 such that when a manual force is applied to the actuator 310, the insertion portion of the sensor 101 is inserted through the skin layer of the user.

In certain embodiments, the mounting unit body 212 is provided with an adhesive which is disposed on the lower surface 216 (FIG. 2) of the mounting unit body 212. In such embodiments, a releasable liner may be provided on the adhesive. Removal of the releasable liner, and pressing the lower surface 216 of the mounting unit body 212 against the skin of the user adheres the mounting unit body 212 to the skin of the user prior to the sensor 101 being inserted.

In one aspect, once the sensor 101 has been inserted into the skin of the user, the sensor insertion mechanism 300 is removed from the mounting unit body 212. Once the sensor insertion mechanism 300 has been removed, a transmitter 102, is removeably attached to the upper surface 214 of the mounting unit body 212 such as shown in FIG. 4. In certain embodiments, transmitter contacts 204, 206, 208 (FIG. 2) disposed on the transmitter unit 102 interface or are otherwise operatively coupled with the respective contacts 230, 232, 234 disposed or otherwise secured on the upper surface 214 of the mounting unit body 212. In another embodiment, the contacts 204, 206, 208 of the transmitter unit 102 can make electrical contact directly with the contacts 220′, 222′, 224′ that are disposed directly on the sensor 101 while the transmitter unit 102 is secured to the mounting unit body 212. In still yet other embodiments, when the transmitter unit 102 has been secured to the upper surface 214 of the mounting unit body 212, the body of the sensor 101 maintains a planar configuration such that the overall profile of the mounting unit assembly 200 is minimized. For example, the sensor 101 can be permanently affixed to the mounting unit body 212 such that the position of the sensor 101 and the flatness of the sensor are maintained. Additionally, as placement of the sensor (relative to contact with the transmitter) is not determined by an insertion mechanism, the contact between the sensor 101 and the transmitter unit 102 is more reliable. Additionally, placing the electronic circuitry on the mounting unit body 212 allows for a reduced transmitter size and weight, which can lower the potential for accidental removal by being bumped, scraped, or hooked on an object during wear.

In certain embodiments, sensor 101, mounting unit body 212, and transmitter unit 102 remain in place on the user's body for a predetermined period, (e.g., three to seven days). After expiration of the predetermined period, the sensor 101 and mounting unit body 212 are removed and discarded while the transmitter unit 102 may be reused.

As discussed above, in certain embodiments, the transmitter unit 102 is disposed upon the upper surface 216 of the mounting unit body 212 such as shown in FIG. 4. In this manner, the mounting unit body 212 may also be configured to include a power source 510 such as, for example, a battery. Thus, in one aspect the transmitter unit 102 does not require a separate power source within its own housing which may further reduce the profile of the transmitter unit 102. For example, although not shown, traces extending from the battery can power the transmitter unit 102. In this manner, the transmitter unit 102 can be configured to have one or more pins or protrusions (e.g., conductive polymer or metal) to contact the battery disposed in the mounting unit body 212 to establish an electrical connection. The battery can be molded into the mounting unit body 212 or can be mounted into the mount and include sockets or vias for connection to the transmitter unit 102. In another embodiment, the battery may be removable and/or rechargeable such that when the mounting unit body 212 and sensor 101 is discarded, the battery may be reused in a subsequent mounting unit body 212 and sensor 101 combination.

In the manner described above, a mounting unit assembly may be provided that includes a body including one or more contacts, one or more conductive traces, and a sensor disposed on or in the mounting unit assembly. In certain embodiments, the one or more contacts and one or more conductive traces can be surface mounted, etched, printed, or tape applied to the mounting unit surface. Alternatively, the electronics can be captured or embedded between laminates or layers that are used to form the mounting unit body. As another alternative, the electronics can be insert molded or impregnated into the mounting unit body during molding. For example, the electronic circuitry can be provided an adhesiveless construction to provide a thinner, more flexible circuit. Because the sensor and corresponding sensor electronics are coupled to the mounting unit to form a single unitary structure, placement of a portion of the sensor under the skin of the user will not cause the sensor to become misaligned or skewed in the user's skin during or after the sensor insertion process. Further, as the sensor electronics are disposed directly on the mounting unit, connection between the contact pins of a transmitter and the sensor electronics may be more reliable as the contacts between the transmitter and sensor are not dependent on sensor alignment. Further, as electronic circuitry associated with the sensor is disposed directly on the mounting unit and not in the transmitter, the overall profile of the transmitter is reduced.

In certain aspects of the present disclosure, a mounting unit assembly may include a mounting unit including first and second opposing surfaces, wherein one or more contacts and one or more conductive traces are disposed directly on the first surface and wherein the second surface is adapted to attach to a user, and a sensor integrally formed with the mounting unit such that the one or more conductive traces extend to one or more electrodes of the sensor.

In certain embodiments, the mounting unit and the sensor may define a single unitary structure.

In certain embodiments, the one or more electrodes of the sensor may be formed of gold.

In certain embodiments, the gold electrodes may be formed from a laser ablation technique.

In certain embodiments, an insertion portion of the sensor may extend from mounting unit.

In certain embodiments, the insertion portion of the sensor may be flexible.

In certain embodiments, the insertion portion of the sensor may extend downwardly from the mounting unit body to define an angled sensor.

Certain embodiments of the present disclosure may further include a transmitter including a body having one or more transmitter contacts disposed thereon.

In certain embodiments, the transmitter may be operatively coupled to the first surface of the mounting unit body, and further wherein the transmitter contacts interface with the one or more contacts disposed on the mounting unit body.

In certain embodiments, the sensor may be embedded, etched, or molded into the first surface of the mounting unit.

In certain embodiments, the conductive traces or contacts may be embedded, etched, or molded into the first surface of the mounting unit.

In certain embodiments, the conductive traces or contacts may be formed from a flexible ribbon disposed on mounting unit body.

Certain embodiments may include a sensor introducer mechanism removeably coupled to the mounting unit body.

In certain embodiments, the sensor introducer mechanism may include an introducer sharp configured to retain at least a portion of the sensor prior to sensor insertion and release the at least the portion of the sensor when the sensor is implanted into the user.

In certain embodiments, the introducer sharp may comprise a spring.

In certain aspects of the present disclosure, a mounting unit assembly may include a mounting unit having first and second surfaces, wherein one or more contacts and one or more conductive traces are disposed directly on the first surface and wherein the second surface is adapted to removeably attach to a user, a sensor integrally disposed within the mounting unit, and a transmitter including one or more contacts integrally disposed in a surface of the transmitter, wherein the contacts of the transmitter interface with the contacts of the mounting unit body when the transmitter is operatively coupled to the first surface of the mounting unit.

In certain embodiments, the transmitter may be powered by a power source associated with the mounting unit.

In aspects of the present disclosure, a mounting unit assembly may include a mounting unit comprising a body having first and second opposing surfaces, wherein the first surface is adapted to detect and monitor an analyte present in a biological fluid and the second surface adapted to attach to a user, an insertion mechanism including a housing, wherein the housing is removeably attached to the mounting unit, an introducer sharp movably disposed within the housing, and an actuator for urging the introducer sharp in an insertion direction, wherein the introducer sharp is configured to releasably receive at least a portion of the first surface of the mounting unit.

In certain embodiments, the first surface of the mounting unit may comprise a sensor integrally formed within the mounting unit.

In certain embodiments, the actuator may include a spring to urge the introducer sharp to an insertion position.

In certain embodiments, the introducer sharp may be formed from a spring.

In certain embodiments, the introducer sharp may be automatically retracted within the housing after the at least the portion of the first surface of the mounting unit is inserted into a skin layer of a user.

Certain embodiments may include a transmitter configured to engage the mounting unit after removal of the insertion mechanism.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

What is claimed is:

1. A mounting unit assembly comprising:
a mounting unit including first and second opposing surfaces, wherein one or more contacts and one or more conductive traces are disposed directly on the first surface and wherein the second surface is adapted to attach to a user; and
a sensor integrally formed with the mounting unit such that the one or more conductive traces extend to one or more electrodes of the sensor.

2. The mounting unit assembly of claim 1, wherein the mounting unit and the sensor define a single unitary structure.

3. The mounting unit assembly of claim 1, wherein the one or more electrodes of the sensor are formed of gold.

4. The mounting unit assembly of claim 3, wherein the gold electrodes are formed from a laser ablation technique.

5. The mounting unit assembly of claim 1, wherein an insertion portion of the sensor extends from mounting unit.

6. The mounting unit assembly of claim 5, wherein the insertion portion of the sensor is flexible.

7. The mounting unit assembly of claim 5, wherein the insertion portion of the sensor extends downwardly from the mounting unit body to define an angled sensor.

8. The mounting unit assembly of claim 1, further comprising a transmitter including a body having one or more transmitter contacts disposed thereon.

9. The mounting unit assembly of claim 8, wherein the transmitter is operatively coupled to the first surface of the mounting unit body, and further wherein the transmitter contacts interface with the one or more contacts disposed on the mounting unit body.

10. The mounting unit assembly of claim 1, wherein the sensor is embedded, etched, or molded into the first surface of the mounting unit.

11. The mounting unit assembly of claim 1, wherein the conductive traces or contacts are embedded, etched, or molded into the first surface of the mounting unit.

12. The mounting unit assembly of claim 1, wherein the conductive traces or contacts are formed from a flexible ribbon disposed on mounting unit body.

13. The mounting unit assembly of claim 1, further including a sensor introducer mechanism removeably coupled to the mounting unit body.

14. The mounting unit assembly of claim 13, wherein the sensor introducer mechanism includes an introducer sharp configured to retain at least a portion of the sensor prior to sensor insertion and release the at least the portion of the sensor when the sensor is implanted into the user.

15. The mounting unit assembly of claim 14, wherein the introducer sharp comprises a spring.

16. A mounting unit assembly comprising:
a mounting unit having first and second surfaces, wherein one or more contacts and one or more conductive traces are disposed directly on the first surface and wherein the second surface is adapted to removeably attach to a user;
a sensor integrally disposed within the mounting unit; and
a transmitter including one or more contacts integrally disposed in a surface of the transmitter, wherein the contacts of the transmitter interface with the contacts of the mounting unit body when the transmitter is operatively coupled to the first surface of the mounting unit.

17. The mounting unit assembly of claim 16, wherein the transmitter is powered by a power source associated with the mounting unit.

18. The mounting unit assembly of claim 16, wherein the sensor is embedded, etched, or molded into the first surface of the mounting unit.

19. The mounting unit assembly of claim 16, wherein the conductive traces or contacts are embedded, etched, or molded into the first surface of the mounting unit.

20. The mounting unit assembly of claim 16, wherein the conductive traces or contacts are formed from a flexible ribbon disposed on mounting unit body.

21. A mounting unit assembly comprising:
a mounting unit comprising a body having first and second opposing surfaces, wherein the first surface is adapted to detect and monitor an analyte present in a biological fluid and the second surface adapted to attach to a user;
an insertion mechanism including a housing, wherein the housing is removeably attached to the mounting unit;
an introducer sharp movably disposed within the housing;
and an actuator for urging the introducer sharp in an insertion direction, wherein the introducer sharp is configured to releasably receive at least a portion of the first surface of the mounting unit.

22. The mounting unit assembly of claim 21, wherein the first surface of the mounting unit comprises a sensor integrally formed within the mounting unit.

23. The mounting unit assembly of claim 21, wherein the actuator includes a spring to urge the introducer sharp to an insertion position.

24. The mounting unit assembly of claim 21, wherein the introducer sharp is formed from a spring.

25. The mounting unit assembly of claim 21, wherein the introducer sharp is automatically retracted within the housing after the at least the portion of the first surface of the mounting unit is inserted into a skin layer of a user.

26. The mounting unit assembly of claim 21, further comprising a transmitter configured to engage the mounting unit after removal of the insertion mechanism.
